(19) United States
(12) Patent Application Publication
(10) Pub. No.: US 2005/0147734 A1
(43) Pub. Date: Jul. 7, 2005

(54) Method and system for coating tubular medical devices

(76) Inventors: Jan Seppala, Maple Grove, MN (US); Jeffrey Sewell, Brooklyn Park, MN (US)

Correspondence Address: KENYON & KENYON, 1500 K STREET, N.W., SUITE 700, WASHINGTON, DC, 20005, US

(21) Appl. No.: 10/752,021

(22) Filed: Jan. 7, 2004

(51) Int. Cl. A61L 2/00; B05D 3/00; B05D 5/12

(52) U.S. Cl. 427/2.1; 427/110; 118/300; 118/324

(57) ABSTRACT

A system and method for application of therapeutic and protective coatings to multiple tubular medical devices in a high volume production process. One or more tubular medical devices, such as stents, are placed on a coating-absorbent core, and coating is applied to the device(s), for example, as when the device-carrying core is passed through an extrusion coating machine to apply the coating in a uniform manner. Once coated, the medical device(s) may be quickly and efficiently removed from the core by causing the core diameter to decrease, such as by applying elongating tension to the core to cause the core diameter to radially contract, thereby allowing the coated device(s) to be simultaneously freed from the core. Improved coating uniformity, increased coated device removal ease and minimized bridging of openings in the tubular medical device may be obtained with a core that absorbs excess coating.

### FIELD OF THE INVENTION

The present invention is directed to the field of applying therapeutic and protective coatings to tubular medical devices, such as stents.

### BACKGROUND

Medical implants are used for innumerable medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Such localized delivery of therapeutic agents has been proposed or achieved using medical implants which both support a lumen within a patient's body and place appropriate coatings containing absorbable therapeutic agents at the implant location. Examples of such medical devices include stents, stent grafts, vascular grafts, catheters, guide wires, balloons, filters (e.g., vena cava filters), intraluminal paving systems, implants and other devices used in connection with drug-loaded polymer coatings. Such medical devices are implanted or otherwise utilized in body lumina and organs such as the coronary vasculature, esophagus, trachea, colon, biliary tract, urinary tract, prostate, brain, and the like.

The delivery of stents is a specific example of a medical procedure that may involve the deployment of coated implants. Stents are tube-like medical devices designed to be placed within the inner walls of a lumen within the body of a patient. Stents typically have thin walls formed from a lattice of stainless steel, Tantalum, Platinum or Nitinol alloys. The stents are maneuvered to a desired location within a lumen of the patient's body, and then typically expanded to provide internal support for the lumen. Stents may be self-expanding or, alternatively, may require external forces to expand them, such as by inflating a balloon attached to the distal end of the stent delivery catheter.

Where a stent is to be coated, care must be taken during its manufacture to ensure the coating is correctly applied and firmly adherent to the stent. When the amount of coating is insufficient or is depleted through stripping of poorly adherent coating during manufacture or deployment within the patient's body, the implant's effectiveness may be compromised, and additional risks may be inured into the procedure. For example, when the coating of the implant includes a therapeutic, if some of the coating were removed during deployment, the therapeutic may no longer be able to be administered to the target site in a uniform and homogenous manner. Thus, some areas of the target site may receive high quantities of therapeutic while others may receive low quantities of therapeutic. Similarly, if the therapeutic is ripped from the implant it can reduce or slow down the blood flowing past it, thereby, increasing the threat of thrombosis or, if it becomes dislodged, the risk of embolisms. In certain circumstances, the removal and reinsertion of the stent through a second medical procedure may be required where the coatings have been damaged or are defective.

The mechanical process of applying a coating onto a stent may be accomplished in a variety of ways, including, for example, spraying the coating substance onto the stent, so-called spin-dipping, i.e., dipping a spinning stent into a coating solution to achieve the desired coating, and electrohydrodynamic fluid deposition, i.e. applying an electrical potential difference between a coating fluid and a target to cause the coating fluid to be discharged from the dispensing point and drawn toward the target. In these prior stent coating systems, the stents typically are coated on all surfaces. For example, with a coating spray application system, the relatively open lattice structure of the stent permits a coating spray to pass through the open areas and coat the inner surfaces of the stent. Similarly, with a spin-dipping stent coating system, all the surfaces of the stent, interior and exterior, are exposed to the coating fluid upon immersion into the coating bath.

In the typical stent deployment, the outside surface of the stent contacts the vessel wall, and therefore, ordinarily, only the outside surface of the stent needs to be coated. Further, in some instances, it may be desired that there is no significant exposure of the coating material to the bloodstream, and therefore it would be desirable to not have any of the coating on the interior surfaces of the stent. Additionally or alternatively, it is desirable to coat only the outside surface of the stent to avoid excessive use of expensive coating agents and/or to leave the inside surface of the stent uncoated to minimize the risk of slippage on the delivery device.

A further disadvantage of the prior coating approaches is their individual handling and coating of each stent in a sequential manner, i.e., they typically are individually placed onto a stent holding mechanism, coated, then removed from the stent holder before the next stent is coated. Such individual handling further contributes to undesirably long stent coating production cycle times.

Thus, there is a need for a method and system for applying a high quality coating on the exterior surfaces of a stent while preventing coating application on interior stent surfaces, and accomplishing these objectives while maintaining low coated stent production cycle times, and, thus, high coated stent production rates.

### SUMMARY OF THE INVENTION

The present invention is directed to a method and system for overcoming one or more of the foregoing disadvantages. Specifically, in one embodiment, there is a provided a method and system in which a core is placed through the longitudinal centers of a plurality of tubular medical devices, such as stents, and the core carrying the medical devices is passed through a coating extrusion die where a coating is applied to the medical devices. Because the tubular medical devices and the cylindrical core are sized to provide a frictional fit between the devices' inner surfaces and the outer diameter of the core, the core effectively masks the devices' inner surfaces from receiving coating during the extrusion process. Thus, the coated medical devices may have coating adhering only on their outer surfaces and to the side edges of any openings through the medical devices. The coated medical devices are then removed from the core for further processing by causing the outer diameter of the core to be reduced and disengage from the devices. The core is desirably coating-absorbent, and, therefore, it may wick excess coating away from any openings in the tubular medical device to assist in preventing “webbing” or “bridging,” i.e., the formation of coating films across such openings.

The foregoing method and system is amenable to a number of variations. For example, the coating may be allowed to dry before the core is removed, thereby minimizing the potential for wet coating to flow onto previously masked surfaces when the core is removed, or the devices may be immediately removed from the core and transferred to drying stations while the coating dries.

In alternative embodiments, the core may be passed through only one medical device and/or other coating mechanisms may be used to coat the device(s) mounted on the core. For example, rather than being passed through a coating extrusion die, the core-mounted devices may be spray-coated.

Because the present invention permits the simultaneous handling and processing of multiple medical devices on a single device-carrying core, high production rates may be maintained while the coating extrusion die provides the desired uniform, high quality coating on the tubular medical devices.

### BRIEF DESCRIPTION OF THE DRAWINGS

The foregoing and further objects, features and advantages of the invention will become apparent from the following description of preferred embodiments with reference to the accompanying drawings, wherein like numerals are used to represent like elements and wherein:

FIG. 1 is an illustration of a core with a plurality of stents mounted thereon in accordance with an embodiment of the present invention.

FIG. 2 is a schematic illustration of the coating of tubular medical devices as the medical devices carried on the core are drawn though a coating extrusion die in accordance with an embodiment of the present invention.

FIG. 3 illustrates the coated devices with the core at a reduced diameter in accordance with an embodiment of the present invention.

### DETAILED DESCRIPTION

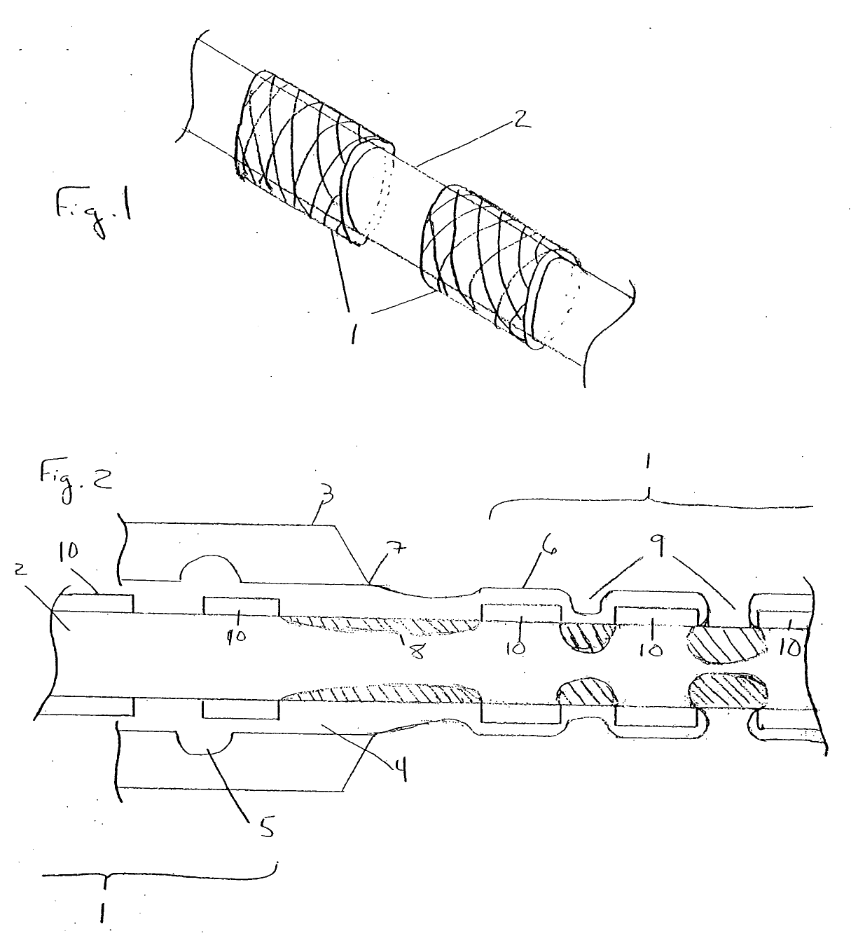

FIG. 1 illustrates a plurality of tubular medical devices (in this embodiment, a plurality of stents 1) which are to receive a coating of a therapeutic material, where the stents 1 have been placed on core 2. Stents 1 are generally cylindrical in shape, and may be in the form of a lattice of a material such as stainless steel, Tantalum, Platinum or Nitinol alloys. A lattice configuration permits stents 1 to radially expand (as during implantation in a patient) or to radially contract (as when the stent is crimped, for example, onto a balloon catheter prior to delivery into a patient's body). The ability of stents 1 to be radially compressed permits adjustment of their inner diameters during placement onto core 2, if necessary, to ensure sufficient frictional engagement between the stents and the core in order to minimize the potential for undesired stent movement along core 2. For example, once stents 1 have been loaded onto core 2, their inner diameter may be reduced by mechanical processes, such as lightly crimping individual stents or passing the stent-loaded core through a sizing die sized to provide the desired stent diameter reduction. Alternatively, the core may be constructed such that it can be in a reduced diameter for loading of the stents and then released or brought to a larger diameter to engage the stents. Sufficient engagement friction is desired to discourage the stents from sliding along the core during handling or coating processes, and therefore to avoid having the stents undesirably close together and possible uneven coating application, for example, as may occur if the ends of two stents were abutting one another.

The core 2 upon which the plurality of stents 1 are carried may comprise a variety of materials and configurations, as long as it provides a substrate which retains the plurality of medical devices as they receive their coating, and then readily releases the plurality of medical devices following application of the coating. In the present embodiment, the core is an absorbent polymer, specifically a cellulose rod that: (i) offers sufficient friction on its outer surface to minimize motion of the plurality of stents placed thereon; (ii) absorbs excess coating material which comes in contact with its outer surface; and (iii) when placed under tension, elongates and reduces in diameter, allowing the plurality of stents to be freely removed from the core. Alternative embodiments of the core include a cylindrical tube rather than a solid rod and alternative geometric shapes rather than a circular cross-section, such as a square or other polygon whose corners contact the inner surface of the tubular medical devices where complete masking of the inner surface of the stents is not necessary. The core may also be composed of alternative materials, such as an absorbent paper or other fibrous material.

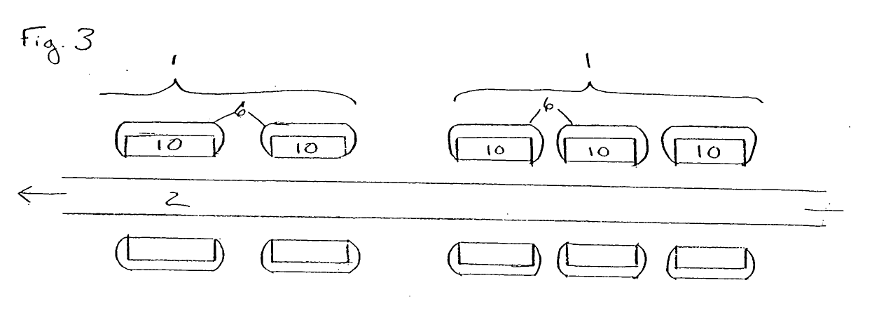

The method of tubular medical device coating in accordance with the present invention is as follows. As illustrated in the cross-sectional view in FIG. 2, core 2 carrying the plurality of stents 1 is fed into an extrusion or slot coating machine 3 through an entry port (not shown). As the stent-carrying core passes though slot 4, the stents are carried past annular coating introduction aperture 5, where coating material 6 is dispensed to apply a continuous layer of coating material over stents 1 and core 2. Slot 4 is sized to provide a uniform coating thickness over stents 1 as they are extruded through coating machine 3 and emerge from outlet 7. In the present embodiment, with a stent outer diameter on the order of 1 to 3.5 mm and a coating with a viscosity on the order of 100 to 100,000 centipoise, a uniform extruded coating may be obtained with an outlet 7 inner diameter of approximately 0.25 mm greater than the outer diameter of the stent. Suitable extrusion processing equipment capable of use with the present invention can be obtained, for example, from C. W. Brabender, South Hackensack, N.J. 07606.

As core 2 passes through slot 4, receives coating material 6 and emerges from coating machine 3, the core begins to absorb the coating material directly in contact with its outer diameter, both in the areas 8 between adjacent stents 1, and in regions 9 under openings in the lattice structure of stents 1 between stent struts or elements 10. The amount of coating material absorbed into core 2 increases the longer the coating is in contact with the core. This is illustrated in FIG. 2, where excess coating material in the inter-stent regions 8 between adjacent stents 1 is being drawn into core 2, and excess coating material over openings in the stent lattice between stent struts or elements 10 is being absorbed by core 2 in regions 9, and the amount of excess coating material absorbed by core 2 increases the farther core 2 extends beyond outlet 7. Because core 2 draws the excess coating material away from the stent lattice openings, core 2 assists in minimizing “webbing” or “bridging,” i.e., the formation of a film of coating material across the lattice openings. The coated stents thus have a uniform thickness coating on their outer surfaces, which may include the sides of the lattice elements, while the stents remain uncoated on their inner diameter surfaces.

Once the core 2 with plurality of stents 1 have been coated, the stents may be allowed to dry on the core by either natural or accelerated means (such as forced air circulation), or the stents may be immediately removed from core 2 and placed on drying stations, such as a series of drying mandrels. With either approach to drying, the plurality of coated stents 1 may be rapidly and efficiently removed from core 2 for further processing and packaging. As illustrated in FIG. 3, when end portions of core 2 are grasped longitudinally and the core is placed under tension, core 2 elongates and its outer diameter is reduced. The reduction in outer diameter draws the outer surface of core 2 radially inward and out of frictional engagement with the inner surfaces of the plurality of stent elements 10, thus allowing the plurality of stents 1 to be rapidly released from core 2 without the need for individual, sequential stent handling. The simultaneous group processing of a plurality stents on each core permits substantially increased coated stent production rates.

As noted above, in the present embodiment core 2 is an elastic polymer. Other materials and diameter-reduction techniques may be employed as long as the plurality of stents 1 are freed for removal from the core. For example, core 2 might comprise a non-reusable, non-elastic material that is permanently deformed into a reduced diameter by the application of longitudinal tension, for example, a spiral-wound paper tube which, when pulled by the ends, elongates and decreases in diameter to free the coated medical devices. Alternatively, rather than applying longitudinal tension to core 2 to reduce core diameter, an inflatable core may be used to hold the stents, and then deflated to obtain the desired core diameter reduction to release the stents; such an inflatable core may be provided with an absorbent outer coating to absorb excess coating material 6 if the inflatable core is insufficiently absorptive itself.

With regard to the coatings discussed above, the term “therapeutic agent” as used herein includes one or more “therapeutic agents” or “drugs.” The terms “therapeutic agents” and “drugs” are used interchangeably herein and include pharmaceutically active compounds, nucleic acids with and without carrier vectors such as lipids, compacting agents (such as histones), virus (such adenovirus, andenoassociated virus, retrovirus, lentivirus and α-virus), polymers, hyaluronic acid, proteins, cells and the like, with or without targeting sequences. Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitorfurantoin; anesthetic agents such as lidocaine, bupivacaine, and ropivacaine; nitric oxide (NO) donors such as lisidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and A, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Coatings used with the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The polymer is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer may be hydrophilic or hydrophobic, and may be selected, without limitation, from polymers including, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS) copolymers, styrene-isoprene-styrene (SIS) copolymers, styrene-butadiene-styrene (SBS) copolymers, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collage and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

While the present invention has been described with reference to what are presently considered to be preferred embodiments thereof, it is to be understood that the present invention is not limited to the disclosed embodiments or constructions. On the contrary, the present invention is intended to cover various modifications and equivalent arrangements. In addition, while the various elements of the disclosed invention are described and/or shown in various combinations and configurations, which are exemplary, other combinations and configurations, including more, less or only a single embodiment, are also within the spirit and scope of the present invention.

1. A method for applying a coating to a tubular medical device, comprising the steps of:
providing a core carrying at least one tubular medical device, wherein the core passes through a longitudinal center of the at least one tubular medical device;
applying coating to the at least one tubular medical device;
reducing the diameter of the core; and
removing the at least one tubular medical device from the core.

2. The method of claim 1, wherein the at least one tubular medical device is a stent.

3. The method of claim 1, wherein the step of applying coating comprises drawing the core carrying the at least one tubular medical device through a coating extrusion die.

4. The method of claim 3, wherein the core absorbs coating which contacts the core.

5. The method of claim 4, wherein the core is one of a resilient polymer cylinder, a spiral-wound paper cylinder, a cylindrical tube comprising an coating-absorbent outer covering over a non-absorbent tube and an inflatable tube.

6. The method of claim 5, wherein the step of reducing the diameter of the core comprises longitudinally stretching the core.

7. The method of claim 5, wherein the step of reducing the diameter of the core comprises deflating the inflatable tube.

8. The method of claim 1, further comprising, prior to the step of removing the at least one tubular medical device from the core, the step of:
drying the coating on the at least one tubular medical device.

9. The method of claim 1, further comprising, after the step of removing the at least one tubular medical device from the core, the step of:
transferring the coated at least one tubular medical device to a coating drying station.

10. A system for coating a tubular medical device, comprising:
a core for insertion through the longitudinal center of, and carrying, at least one plurality of tubular medical devices; and
a coating applicator,
wherein, when the core carrying the at least one tubular medical device is passed through the coating applicator, coating is applied to the at least one tubular medical device.

11. The system of claim 10, wherein the at least one tubular medical device is a stent.

12. The system of claim 10, wherein the coating applicator is a coating extrusion die.

13. The system of claim 12, wherein the core absorbs coating which contacts the core.

14. The system of claim 13, wherein the core is one of a resilient polymer cylinder, a spiral-wound paper cylinder, a cylindrical tube comprising an coating-absorbent outer covering over a non-absorbent tube and an inflatable tube.

15. The system of claim 14, wherein the core outer diameter is reduced when the core is stretched longitudinally.

16. The system of claim 14, wherein the core outer diameter is reduced when the core is an inflatable tube and the inflatable tube is deflated.
